(19) United States
(12) Patent Application Publication
(10) Pub. No.: US 2006/0025579 A1
(43) Pub. Date: Feb. 2, 2006

(54) Process for extraction of biological material

(75) Inventors: Wolfgang Riedl, Weil Am Rhein (DE); Ulrich Buhlmann, Biel-Benken (CH); Dirk De Bruyn Ouboter, Ettingen (CH)

Correspondence Address: BIRCH STEWART KOLASCH & BIRCH, PO BOX 747, FALLS CHURCH, VA, 22040-0747, US

(73) Assignee: Kuhni AG, Allschwil (CH)

(21) Appl. No.: 11/159,362

(22) Filed: Jun. 23, 2005

(30) Foreign Application Priority Data
Jun. 25, 2004 (EP) 04405395.7

(51) Int. Cl. C07K 1/14

(52) U.S. Cl. 530/412

(57) ABSTRACT

In a process for extraction of biological material, in particular of proteins or peptides, from a first aqueous solution (1) to a second aqueous solution (2), a porous membrane (3) is arranged between the aqueous solutions (1, 2). At least one of the aqueous solutions (1) is now modified by addition of a biocompatible and surface-active agent in such a way that the wetting behaviour of the two liquid phases on the membrane and in the pores of the latter differs significantly. In this way, membrane extraction of biological material from a first aqueous solution (1) to a second aqueous solution (2) is made possible in a simple and efficient manner. The process is easy to carry out and can be used on an industrial scale.

### TECHNICAL FIELD

The invention relates to a process for extraction of biological material, in particular of proteins or peptides, from a first aqueous solution to a second aqueous solution, a porous membrane being arranged between the aqueous solutions. The invention further relates to the use of a biocompatible agent in a process for extraction of biological material.

### PRIOR ART

Isolation and purification of biological material, for example of proteins, peptides or other substances produced chemically, biologically or by biotechnology or genetic engineering and having a biological interaction (“biological activity”) with plants and/or organisms, generally require multi-stage separation processes and entail considerable costs.

Since such products are often stable only in aqueous solutions within narrow limits of temperature, pH value and ionic strength, the process of extraction by aqueous two-phase systems (ATPS), which has been extensively researched in biotechnology, often presents a suitable recovery process. Information on this can be found, for example, in C. Grossmann, “Untersuchungen zur Verteilung von Aminosäuren und Peptiden auf wässrige Zwei-Phasen-Systeme” [Investigations into the distribution of amino acids and peptides on aqueous two-phase systems], Dissertations Druck Darmstadt, Kaiserslautern, 2002, or in J. Brenneisen “Zur Verteilung von Proteinen auf wässrige Zwei-Phasen-Systeme” [On the distribution of proteins on aqueous two-phase systems], published by Der Andere Verlag, Osnabrück, 2001.

The production of ATPS involves mixing aqueous solutions of two polymers which are soluble in water but are not miscible with one another. Two phases form after the mixing. The ATPS which has been most extensively investigated to date is polyethylene glycol (PEG)/dextran. Both polymers are infinitely miscible with water. However, if they are added together, a phase separation takes place starting from a certain polymer concentration, with formation of a PEG-rich phase and a dextran-rich phase. The same phenomenon can also be achieved with other substance pairings, for example PEG/phosphate buffer.

The advantage of the ATPS lies in the water-like conditions of the two phases. These often comprise more than 80% water. The biologically active components can be extracted gently, without losing their activity (e.g. by denaturation as a consequence of so-called unfolding). With suitable buffer systems, even the pH of the phases can be set at a desired value, as a result of which denaturation (e.g. in a strongly acid medium) can again be effectively avoided. In addition to the pH, other factors such as the concentration of the phase-forming components, temperature, polymer molecular weight, salts, etc., can be used to optimally adapt these multi-variable systems to the separation objective. Moreover, with ATPS of this kind, the cell residues which often disrupt fermentation processes can be removed separately because they accumulate preferentially in one of the two phases.

However, the disadvantage of aqueous two-phase systems is that differences in the physical properties of the two phases are scarcely discernible. In particular, the difference in density of the two aqueous phases is very low (less than 60 kg/m³) because of the high water content (in most cases over 80% by weight). A sufficiently great difference in density (typically more than 80 kg/m³) is necessary, however, to ensure that the two phases can be separated completely in the gravity field. Where there is less difference in density, as is the case in the extraction of useful components using aqueous two-phase systems, centrifugation must therefore generally be performed, see for example K. Sattler, “Thermische Trennverfahren” [Thermal separation processes], published by Verlag VCH, Weinheim, 1995.

However, because of the high speeds of rotation needed to generate high centrifugal forces, the centrifugation technique is onerous and expensive. With large volumes, that is to say in industrial application, the expense increases because these volumes must be made available in the rotating drum, as a result of which the mass to be moved is substantially increased. Large centrifuges are expensive to acquire and to maintain, however, and they require a great deal of energy and necessitate safety measures. Moreover, it is often the case that a maximum of one theoretical separation stage can be effected by means of centrifugation. In ATPS processes for cleaning and isolation of biological material, however, several separation stages are sometimes required. The advantages of extraction by means of ATPS are thus greatly compromised (in particular in large-scale operation) by the required use of centrifugation.

In the extraction of substances from a first liquid phase to a second liquid phase, it is also known to immobilize the phase interface between the two liquid phases in a porous membrane. This generally takes place because one of the two liquid phases wets the membrane and can penetrate into the pores of the latter, whereas the other liquid phase does not wet the membrane or is prevented from penetrating it by the first-mentioned liquid phase which was able first to penetrate into the membrane pores (see T. Melin, R. Rautenbach, “Membranverfahren” [Membrane processes], published by Springer-Verlag, Berlin, 2003). The corresponding technology of membrane-assisted liquid-liquid extraction (also known, inter alia, by the terms “pertraction” or “dispersion-free solvent extraction”) is in principle in direct competition with conventional extraction techniques such as extraction in columns (with or without moving components), with mixer-settlers and centrifugal extractors.

By immobilizing the phase interface in the membrane pores, the following advantageous properties are obtained in principle:
Dispersal of the first phase in the second phase is not necessary, and both phases remain homogeneous. Phase separation after extraction is therefore not required (dispersion-free extraction).
The risk of formation of stable emulsions is greatly limited.
No density difference is needed between the liquid phases.
Continuous operation in countercurrent is possible. In this way, several theoretical separation stages can be achieved in one membrane module.
The phase conditions can be freely selected within wide ranges.
The temperature can be different in the individual phases.
Large specific exchange surfaces can be provided by the membrane, in particular when using capillary and hollow fibre modules (“membrane contactors”, up to 4000 m² per m³ apparatus volume, see T. Melin and R. Rautenbach, “Membranverfahren” [Membrane processes], published by Springer-Verlag, Berlin, 2003).
As long as the wetting properties of both liquids significantly differ from one another, extraction is also possible outside the two-phase area of the substance system.
Scale-up is simple (similar to scale-up in other membrane processes, e.g. ultrafiltration).

Applications of membrane-assisted liquid-liquid extraction were previously based predominantly on organic solvent/water systems. In the literature, the (possible) application of membrane-assisted liquid-liquid extraction is at present chiefly to be found in connection with the cleaning of aqueous waste streams (C. Yun et al., Membrane Solvent Extraction Removal of Priority Organic Pollutants from Aqueous Waste Streams; Ind. Eng. Chem. Res.; 1992; 31(7); 1709-1717), caprolactam production (W. Riedl, “Membrangestützte Flüssig-Flüssig-Extraktion bei der Caprolactam-herstellung [Membrane-assisted liquid-liquid extraction in caprolactam production], published by Shaker Verlag, Aachen, 2003) and process-integrated extraction of simple products of fermentation (G. Frank, K. Sirkar, Alcohol production by yeast fermentation and membrane extraction, Biotech. and Bioeng. Symposium, 1986, 621-631).

Since most of the membranes presently available on the market and suited for membrane-assisted extraction are of a hydrophobic character, in conventional extractions the organic solvent is the wetting liquid and water is the non-wetting liquid.

However, because of their denaturing action, organic solvents cannot be considered for extraction of many biological substances.

Attempts were therefore made to extract biological material by membrane-assisted extraction in the context of aqueous two-phase systems. However, if hydrophobic membranes are used here, as is generally the case, neither of the two aqueous phases wets the membrane.

Dahuron and Cussler describe such attempts at extraction of proteins from aqueous two-phase systems by means of hydrophobic, microporous membranes (Protein Extraction with Hollow Fibers, AlChE Journal (34/1), 1998). Since neither of the two aqueous phases wets the hydrophobic membranes used there, it was attempted to produce the liquid phase contact via the membrane by applying different external pressures to the two liquids. However, problems relating to process stability arose; extraction of cytochrome-e, myoglobin, α-chymotrypsin, catalase and urease in a PEG/phosphate buffer system was in fact possible only by extremely precise adjustment and control of the pressure between inlet and outlet of the membrane module in the two liquid phases. Therefore, safe (long-term) operation on an industrial scale which is also easy to carry out cannot be anticipated with the technique described there.

### DISCLOSURE OF THE INVENTION

The object of the invention is to make available a process belonging to the technical field mentioned in the introduction and permitting extraction of biological material, which process is easy to carry out and can be used on an industrial scale.

The object is achieved by the features of Claim 1. According to the invention, at least one of the aqueous solutions is modified by addition of a biocompatible and surface-active agent in such a way that, in relation to the membrane used, it has a different wettability than the other aqueous solution.

The first aqueous solution with the biological material to be extracted is thus in contact with one surface of the membrane, and the second aqueous solution (the cleaning liquid) is in contact with the other surface of the membrane. Although the solutions are not directly in contact with one another in fluidic terms, the biological material can still pass from one side of the surface to the other side.

The modification of one of the aqueous solutions by addition of the surface-active agent leads to a change in the surface tension of the modified solution and thus to a marked difference in the wetting of the two solutions on the membrane used. The problems known from the prior art and the instability, for example with respect to the pressure of the two phases, are therefore not to be expected.

Because of the biocompatibility of the agent used, there is no denaturation of the biological material contained in the modified aqueous solution or of the biological material extracted. The choice of the agents to be used and their concentration is dependent on the useful substances present in the first aqueous solution, that is to say both the substance to be extracted and also the other biological material which, for example, is to be extracted from the first aqueous solution in further process steps. It is important that none of these useful substances is denatured by the surface-active agent used.

Tests have in fact shown, surprisingly, that, by addition of the agent, membrane extraction of biological material from a first aqueous solution to a second aqueous solution is made possible in a simple and efficient manner. With the process set out here, it is therefore possible for the first time to reliably extract different biological material by means of different aqueous two-phase systems with membrane contactors. To do so, no special demands have to be placed either on the membrane contactors used or on the operating parameters required for the extraction. It is thus possible to benefit both from the advantages of the aqueous two-phase system and from the advantages of membrane extraction.

The tests have also shown, surprisingly, that the addition of the surface-active agent to the one aqueous solution can additionally afford the advantage that the distribution of the biological material, compared to systems without a surface-active component, is positively influenced in favour of a higher concentration in the acceptor phase, which contributes to a heightened degree of extraction and/or a reduction in the separation stages needed for the extraction. If several possible surface-active agents are available for extraction, it is possible to specifically choose the one having the best possible action.

The at least one of the aqueous solutions is preferably modified in such a way that it wets the porous membrane used and can penetrate into the pores of the latter, whereas the other aqueous solution does not wet the membrane and thus does not penetrate into the pores of the latter.

The contact angle between a liquid and the membrane surface can be easily determined and provides a way of measuring the wettability. A liquid with a contact angle to the membrane (irrespective of whether it is hydrophobic or hydrophilic) of at least 90° is regarded as non-wetting liquid, while another liquid with a contact angle of less than 90° is regarded as wetting liquid. By means of the inventive modification of one phase in particular, it is thus ensured that the modified phase preferentially wets the membrane. The modification also has the consequence that one of the solutions can penetrate into the pores of the membrane before the other aqueous solution. Because the contact angle can be measured easily and thus quickly and without too much effort, an extraction can be prepared for by conducting a short series of tests into the effect of different surface-active agents with different concentrations. Based on these preliminary tests, the addition of this agent for the extraction can then be done at the necessary and sufficient concentration.

The one of the aqueous solutions is preferably modified in such a way that a difference between a first contact angle between the first aqueous solution and the surface of the membrane and a second contact angle between the second aqueous solution and the surface of the membrane is at least 5°, preferably at least 10°. The extraction is made easy to carry out because of the difference in the wettability of the two aqueous phases, for which the difference in contact angle provides a measure.

The porous membrane is advantageously hydrophobic. Such membranes are readily available on the market.

In the case of a hydrophobic membrane, the one of the aqueous solutions is preferably modified in such a way that a contact angle between the modified aqueous solution and a surface of the hydrophobic membrane is less than 90°, preferably less than 50°.

The added surface-active agent is preferably a surfactant. The tests carried out have shown that addition of a biocompatible surfactant allows the one of the aqueous solutions to be modified in such a way as to permit unproblematic membrane-assisted extraction.

This is surprising in view of the fact that, in the relevant specialist literature (T. Melin, R. Rautenbach, “Membranverfahren” [Membrane processes], published by Springer Verlag, Berlin, 2003), the presence of surfactants was regarded as critical for the process stability of membrane-assisted liquid-liquid extraction, because these would accumulate on the membrane surfaces and could thus cancel out the different wettability of the two liquid phases. This would lead in extreme cases to phase breakthrough (the expression “incorrect phase formation” is also used at another point), in which one of the two phases passes over into the other.

However, during the series of tests that were carried out, repeated extractions with addition of surfactants were able to be performed for a period of about five months with the same membrane contactor, without incorrect phase formation or instability of the process being observed. Moreover, the contactor was able to be operated with different aqueous two-phase systems, different proteins and also alternately with surfactant and (in comparison tests) without surfactant. The results of repeated tests were in all cases reproducible and the process stability was always assured.

At the end of each test, the membrane contactor was flushed with hot water alone, as is customary, or often also obligatory, in membrane processes (e.g. in ultrafiltration).

Extraction and/or wetting tests were carried out with the surfactants Tween® 20 (CAS 9005-64-5), Triton® X-100 (CAS 9002-93-1) and Brij® 35 P (CAS 9002-92-0). The results confirmed that these three different surfactants can be used successfully in the inventive process for modification of the aqueous phases. This indicates that other biocompatible surfactants can also be used in the context of the invention.

The one of the aqueous solutions is advantageously modified in such a way that a concentration of the surfactant in the modified aqueous solution is at most 2.5% by weight, preferably less than 1.0% by weight. Even such a low concentration is sufficient to bring about the required different wetting of the two liquid phases on the membrane. At the same time, at such low concentrations, there is scarcely any danger of accumulation of these surfactants in and on the membrane matrix, and damage to the useful substances by the surfactant can be effectively avoided.

When using a hydrophilic membrane for the separation, the one of the two aqueous solutions is advantageously modified in such a way that a contact angle between the modified aqueous solution and a surface of the hydrophilic membrane is at least 90°.

The agent chosen for modifying the one aqueous solution is preferably an agent which, in comparison with the other aqueous solution, accumulates very much preferentially in the aqueous solution that is to be modified. This selectivity has the effect that the wettability of the solution to be modified is essentially influenced. The properties of the other solution also then change much less markedly when the other solution comes into contact with the surface-active agent. Correspondingly, a substantial difference in the wettability of the solutions can easily be achieved.

The membrane is preferably a porous flat membrane, capillary membrane or hollow fibre membrane. The process according to the invention can be carried out efficiently using such well-proven and readily available membranes.

A maximum pore diameter of the membrane is advantageously 2 μm. The membrane thus has a microporous structure, so that it is permeable to the biological material that is to be extracted. Depending on the useful substance (biological material) to be extracted, much smaller diameters can also be used, ranging to as little as ca. 0.01 μm and less. With pore diameters of more than 2 μm, a stable extraction process is no longer possible because the channels are so large that the solutions can pass through the pores without being appreciably influenced by the membrane. For this reason, the phase interface can no longer be immobilized in the membrane pore, and the phases no longer remain homogeneous.

The membrane can be made of an organic, inorganic or composite material.

One of the aqueous solutions preferably contains a polymer, in particular PEG (polyethylene glycol) or dextran. A salt, e.g. sodium sulphate (Na₂SO₄) or a phosphate buffer, e.g. a potassium hydrogen phosphate (K₂HPO₄/KH₂PO₄), is preferably dissolved in the other aqueous solution.

In other aqueous two-phase systems which can be used in the context of the invention, the first aqueous solution contains a first polymer, and the second aqueous solution contains a second polymer, the polymers being water-soluble and being immiscible or only slightly miscible with one another. The two polymers are preferably PEG and dextran.

The invention is not limited, however, to the aqueous two-phase systems mentioned. The results of the tests carried out indicate that membrane-assisted liquid-liquid extraction can also be performed safely and with long-term stability using other aqueous two-phase systems which are already known or are still to be developed.

Before or after the extraction, at least one of the aqueous solutions advantageously has its physical, chemical and/or thermodynamic properties altered by means of distillation, by means of membrane processes, flocculation and/or by other suitable separation processes. Such measures, which are known per se, may improve the extraction performance or the quality of the extracted substance.

In the context of the invention, a biocompatible agent is used in a process for extraction of biological material from a first aqueous solution to a second aqueous solution, a porous membrane being arranged between the aqueous solutions, and at least one of the aqueous solutions being modified by addition of the agent in such a way that, in relation to the membrane used, it has a different wettability than the other aqueous solution.

Further advantageous embodiments and combinations of features of the invention will become clear from the following detailed description and from all of the patent claims.

### BRIEF DESCRIPTION OF THE DRAWINGS

In the drawings used to explain the illustrative embodiment:

FIG. 1 shows a schematic representation of the principle of membrane-assisted extraction;

FIGS. 2A and 2B show graphs of the concentration of the proteins extracted in the context of tests, in both phases of an aqueous two-phase system;

FIG. 3 shows a schematic representation of the contact angle of wetting and non-wetting liquids on a surface;

FIG. 4 shows a schematic representation of the equipment used for the membrane extraction tests;

FIG. 5 shows a schematic representation of the membrane contactor used;

FIG. 6 shows a graph of the concentration profile of lysozyme during an extraction test with later addition of the surfactant; and

FIG. 7 shows a schematic representation of the implementation of the process according to the invention.

In the figures, identical parts are in principle provided with identical reference numbers.

### EMBODIMENTS OF THE INVENTION

Conventional Aqueous Two-Phase Systems

From the literature (e.g. C. Grossmann, “Untersuchungen zur Verteilung von Aminosäuren und Peptiden auf wässrige Zwei-Phasen-Systeme” (Investigations into the distribution of amino acids and peptides on aqueous two-phase systems], Dissertations Druck Darmstadt, Kaiserslautern, 2002; J. Brenneisen, “Zur Verteilung von Proteinen auf wässrige Zwei-Phasen-Systeme” [On the distribution of proteins on aqueous two-phase systems], published by Der Andere Verlag, Osnabrück, 2001; J. Rydberg et al., Principles and practices of solvent extraction, Marcel Dekker Inc., New York, 1992, pages 339-347, and H. Walter et al., Methods in Enzymology—Aqueous Two-Phase Systems, Academic Press, San Diego, 1994, volume 228), the following commonly used aqueous two-phase systems (ATPS) are known for example:
Water/PEG 6000/Dextran 500
Water/PEG 6000/K₂HPO₄
Water/PEG 6000/Dextran 70/Na₂SO₄
Water/Dextran 48/PEG 4000
Water/Dextran 48/PEG 4000/phosphate buffer/NaCl
Water/PEG 4000/(K₂HPO₄/KH₂PO₄)

An aqueous two-phase system is produced by mixing aqueous solutions of the stated polymers and salts. Two phases form after the mixing. Since ATPS involves fewer differences in density and greater viscosities than in conventional liquid-liquid extraction, the phase separation in the gravity field takes longer than in the case of systems with organic solvents and water. According to the abovementioned publication by Rydberg et al., times of between five and thirty minutes have to be reckoned with.

Since the density difference of the two phases is low (usually less than 60 kg/m³) on account of the high water content, complete separation of the phases then takes place by centrifugation. This makes the process considerably more expensive and takes up a great deal of time (of the order of thirty minutes), as a result of which the throughput in conventional extractions is considerably reduced.

Membrane-Assisted Extraction

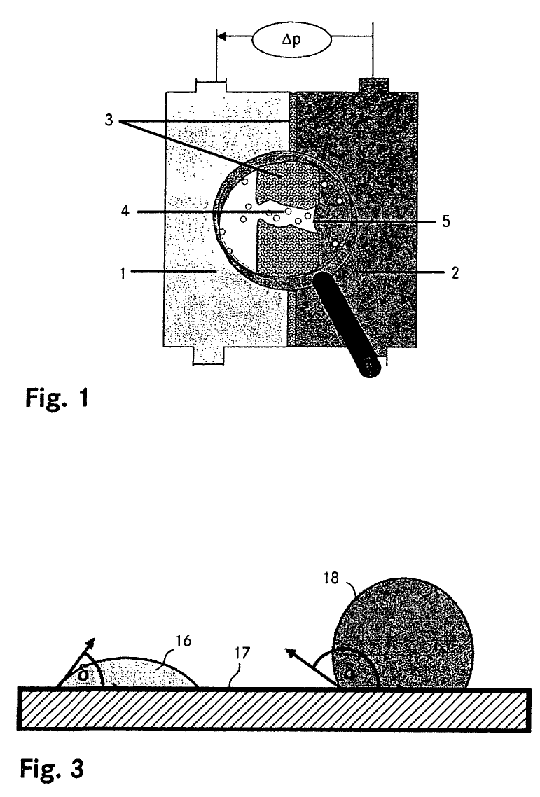

The principle of membrane-assisted extraction is shown in FIG. 1. The two liquid phases 1, 2 are separated from one another by means of a porous membrane 3, and they do not therefore have to be mixed together intensively as is the case in conventional liquid-liquid extractions. Dispersal of one phase in the other liquid phase is therefore not necessary, and the expression “dispersion-free liquid-liquid extraction” is thus also used. Therefore, phase separation (in the gravity field or centrifugal force field) is not needed after the extraction.

The membrane 3 is wetted predominantly by the first phase 1. Since the membrane pores 4 have a small diameter of less than ca. 2 μm and the wall thicknesses of the membrane are small at less than ca. 300 μm, the membrane pores 4 are filled completely with the wetting phase 1 on account of capillarity. The phase interface 5 between the two liquid phases 1, 2 thus forms at the pore outlets of the membrane 3 on that side of the membrane 3 on which the non-wetting liquid phase 2 lies.

To ensure that the wetting phase 1 does not migrate into the non-wetting phase 2, the latter can be subjected to a certain pressure, such that a transmembrane pressure Δp prevails between the two liquid phases 1, 2. In general, pressures of less than 400 mbar are sufficient for this purpose (K. Wecker, “Untersuchungen zum Trennverhalten eines Hohlfaser-moduls bei der Flüssig-Flüssig-Extraktion und Vergleich mit der Leistung einer Packungskolonne” [Investigations into the separating behaviour of a hollow fibre module in liquid-liquid extraction, and comparison with the performance of a packing column], dissertation, University of Erlangen-Nürnberg, Erlangen, 1998). The transmembrane pressure at the same time ensures that the phase interface 5 is immobilized in the membrane pore 4.

According to T. Melin, R. Rautenbach, “Membranverfahren” [Membrane processes], published by Springer-Verlag, Berlin, 2003, and W. Riedl, “Membrangestützte Flüssig-Flüssig-Extraktion bei der Caprolactamherstellung” [Membrane-assisted liquid-liquid extraction in caprolactam production], published by Shaker Verlag, Aachen, 2003, the membrane, in membrane-assisted liquid-liquid extraction, has virtually no selectivity with respect to the components to be extracted. This is because the pore diameters are usually significantly larger than the molecule diameters of the dissolved components. This can, be seen, for example, from the following table, in which molecule diameters of selected dissolved components are listed in comparison with a 0.1 μm membrane pore:

| Substance dissolved | | |
|---|---|---|
| in water | Diameter d [μm] | d_{Pore} (0.1 μm)/d |
| Glucose | 4.44 × 10⁻⁴ | ˜225:1 |
| Caprolactam | <6 × 10⁻⁴ | ˜160:1 |
| β-Dextrin | 8.00 × 10⁻⁴ | ˜125:1 |

As the table shows, the ratio of the membrane pore diameter (0.1 μm) to the diameter of the listed molecules is at least 125:1.

Tests Carried Out

To carry out a first detailed series of tests, an aqueous two-phase system was chosen consisting of polyethylene glycol 4000/phosphate buffer/water with a pH of 7.2. To produce the system, 2000 g of PEG stock solution, consisting of 500 g of PEG and 1500 g of deionized water, and 1800 g of buffer stock solution, consisting of 220 g of potassium dihydrogen phosphate (KH₂PO₄) and 280 g of dipotassium hydrogen phosphate (K₂HPO₄) and 1300 g of deionized water, were weighed into a separating funnel and mixed. Once the phases had separated, they were isolated at room temperature (22° C.). Both still slightly turbid phases were clarified by adding in each case 250 g of deionized water.

Conventional extraction tests were first carried out in the separating funnel. The equilibrium distributions of the proteins bovine serum albumin (BSA, molecular mass 67 kDa) and lysozyme (molecular mass 14.6 kDa) on the aqueous two-phase system were measured at room temperature and at 36° C.

In measuring the distribution of BSA, three different BSA concentrations (1000 mg/system, 1500 mg/system, 2000 mg/system) were investigated in an ATPS. For this purpose, BSA was added in crystalline form to the produced ATPS and was dissolved by gentle shaking. The system was then agitated for three minutes in the separating funnel. The phases separated after just a few minutes, but there was still pronounced turbidity in the two phases. The system was left to settle overnight and was clear the next morning (after ca. 14 hours). The phases were separated at a room temperature of 23° C. and their volumes were measured. The lower phases which had once again become slightly turbid were centrifuged for thirty minutes at 4800 rpm (r=10 cm). After the centrifugation, the phases were still slightly turbid, although a thin upper phase formed. The bottom part was drawn off with a pipette. Some of this sample was examined under a light microscope at a magnification of 40 times. It could be clearly seen that the turbidity originates from small bubbles in the second phase. The bubble diameter was 2.5-9 μm. The samples were again left to stand overnight. On the next day, these samples too were clear, and a thin film of the upper phase could once again be observed on the surface in each case.

The BSA content of the clear samples was determined in duplicate by photospectrometry at 280 nm. Since the BSA concentrations in the PEG phases were below the measurement range, these were calculated from the mass balance.

In parallel with this, the same tests were carried out with an ATPS to which was added 0.55 g of the non-ionic surfactant Tween® 20 (polyethylene glycol sorbitan monolaurate). It was first of all possible to observe, with the naked eye, that the systems with Tween® 20 form significantly more foam than do those without the surfactant.

To examine the distribution of lysozyme on the ATPS, different lysozyme concentrations (250 mg/system, 500 mg/system, 750 mg/system) were again examined in an ATPS without Tween® 20 and in an ATPS with 0.55 g of admixed Tween® 20. Unlike BSA, lysozyme cannot be added in crystalline form to the ATPS, since it forms agglomerates which dissolve only with difficulty. To get round this problem, a 9.7% strength lysozyme solution in water was prepared. This solution replaced part (2.5 g, 5 g or 7.5 g depending on the required protein quantity) of the 10.0 g of water needed in preparing the ATPS. The remainder of the water needed in preparing the ATPS was admixed after the addition of the lysozyme solution.

The rest of the procedure corresponded to that for the systems with BSA. The separating behaviour was also identical. Both phases were centrifuged and analyzed.

A further system with 60 mg of lysozyme was additionally measured at 36° C.; for this purpose the prepared ATPS, with the added protein, was heated in a heating bath to 36° C., shaken for three minutes thereafter, and left overnight at 36° C. to separate. The phases were separated, and the lysozyme content was analyzed by repeat determination.

The following tables show a summary of the analysis results. The tables show in each case the weighed-in quantity of the corresponding protein, whether the surfactant Tween® 20 has been added or not, and the concentrations c in the upper phase (Top) and in the lower phase (Bottom) of the two liquid phases in the separating funnel.

bovine serum albumin (BSA) at room temperature (23° C.):

| Weighed- | | | | |
|---|---|---|---|---|
| in | | | cBSA, | |
| quantity | | cBSA, Top | Bottom | cTop/ |
| (mg) | Tween ® 20 | (mg/ml) | (mg/ml) | cBottom |
| 1000.6 | No | 2.476 | 20.884 | 1:8.4 |
| 1496.7 | No | 1.538 | 32.132 | 1:20.9 |
| 2013.3 | No | 2.059 | 42.755 | 1:20.8 |
| 998.6 | Yes | 0.661 | 22.192 | 1:33.6 |
| 1498.1 | Yes | 0.762 | 32.724 | 1:42.9 |
| 2016.4 | Yes | 1.510 | 43.188 | 1:28.6 |

Lysozyme at room temperature (24° C.):

| Weighed- | | | | |
|---|---|---|---|---|
| in | | | cLYS, | |
| quantity | | cLYS, Top | Bottom | cTop/ |
| (mg) | Tween ® 20 | (mg/ml) | (mg/ml) | cBottom |
| 244.0 | No | 1.2254 | 4.7888 | 1:3.9 |
| 488.7 | No | 2.2300 | 8.5201 | 1:3.8 |
| 735.1 | No | 3.0945 | 14.5150 | 1:4.7 |
| 247.3 | Yes | 1.1847 | 4.9058 | 1:4.1 |
| 490.1 | Yes | 2.0446 | 9.6627 | 1:4.7 |
| 750.0 | Yes | 2.8467 | 15.0180 | 1:5.3 |

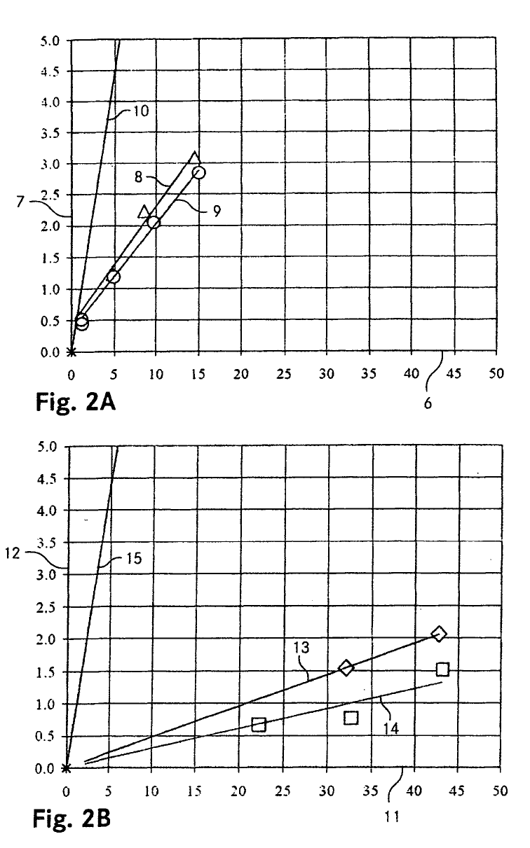

The results are shown in graph form in FIGS. 2A and 2B. FIG. 2A shows the distribution of lysozyme in both phases of the ATPS. Along the horizontal axis 6, the above-cited concentrations in the buffer phase are given in mg/ml, and along the vertical axis 7, in the same units, the cited concentration in the PEG phase. The line 8 connects those results which had been measured without addition of the surfactant, and the line 9 connects the measurement points showing the measured concentrations with addition of the surfactant. For comparison purposes, the straight line 10 indicates the case of an identical concentration in both phases.

In a similar way, FIG. 2B shows the distribution of BSA in both phases. Along the horizontal axis 11, the concentration in the buffer phase is again shown, and along the vertical axis 12 the concentration in the PEG phase, both in mg/ml. The measurement points without addition of surfactant are connected by the line 13, and those with addition of the surfactant are connected by the line 14. The straight line 15, which corresponds to an identical concentration in both phases, once again serves for purposes of comparison.

As can be seen from both figures, both BSA and lysozyme are distributed preferably to the lower, buffer-rich phase. By addition of Tween® 20, the distribution in both proteins is surprisingly shifted in favour of the (lower) buffer phase. Tween® 20 appears to displace both proteins additionally from the (upper) PEG phase, as a result of which the success of extraction can be still further improved if, like here, the buffer phase is chosen as acceptor phase.

Next, preliminary tests for membrane extraction were carried out. Since the wetting properties of the aqueous solutions are important for stable operation of membrane-assisted extraction, the wetting behaviour of the two aqueous solutions of the ATPS was investigated. Processes which require minimal outlay and rapidly provide information on the feasibility of membrane-assisted extraction are the determination of the contact angle of a drop of liquid applied to a surface made of the material of the membrane selected for the extraction, or measurement of the capillary rise when a capillary membrane or hollow fibre membrane is immersed in the liquid phase (W. Riedl, “Membrangestützte Flüssig-Flüssig-Extraktion bei der Caprolactam-herstellung [Membrane-assisted liquid-liquid extraction in caprolactam production], published by Shaker Verlag, Aachen, 2003).

At contact angles of 0°<δ≦90°, a liquid 16 wets the surface 17, while at contact angles 90°<δ<180° a liquid 18 is regarded as non-wetting (FIG. 3). Since the feasibility of membrane-assisted extraction depends above all on there being a significant difference in the wetting behaviour of the two aqueous solutions, it suffices also to note that one of the solutions forms a distinct drop on the membrane, and the other does not.

The measurement of the capillary rise is based on the fact that a wetting liquid rises in a capillary. Since the pore length and also the pore diameter are very small in membranes, it is possible, even in the case of a slight rise, to assume that the membrane is completely wetted.

Precise contact angle measurements were carried out on both phases of the ATPS; the phase drops forming on the membrane surface were recorded photographically from several directions. The angle was then determined from photographs of several drops. As expected, the measurements showed that neither the aqueous PEG phase nor the aqueous buffer phase wets the hydrophobic membrane used (Celgard® 2400), made of polypropylene. A contact angle of 102±4° was determined for the buffer phase, and a contact angle of 105±3° was determined for the PEG phase. Correspondingly, no capillary rise was observed. Continuing tests confirmed that, by admixing the non-ionic surfactant Tween® 20 to the ATPS, the PEG phase is selectively changed such that it wets the membrane used. The contact angles change to 92±4° for the buffer phase and to 47±2° for the PEG phase.

In equilibrium, Tween® 20 in fact accumulates very much preferentially in the PEG phase, so that the wetting properties of the buffer phase are less strongly changed by the surfactant than are those of the PEG phase. It was possible to confirm this by means of solubility tests of Tween® 20 in the employed aqueous solutions of the ATPS components in which it was found that Tween® 20 dissolves in the PEG solution, but forms a separate phase in the buffer solution. This solution behaviour is probably due to the structural relationship between PEG and Tween® 20.

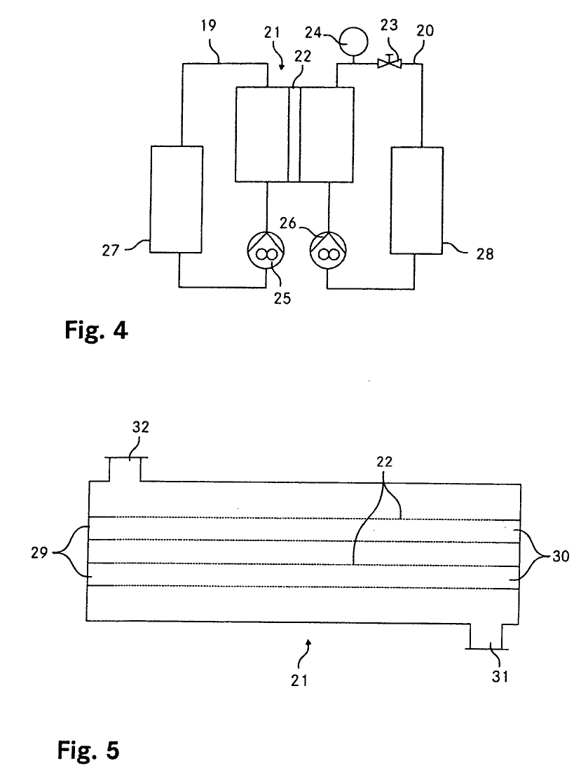

The equipment used for the membrane extraction tests is shown schematically in FIG. 4 and is made up of two independent circuits 19, 20 for the acceptor phase and the donor phase. A membrane contactor 21 with a membrane 22 is incorporated as interface between the two circuits 19, 20. To set up a transmembrane pressure, the circuit 20 of the phase not wetting the membrane 22 is equipped with a control valve 23 and a pressure gauge 24. Temperature, flow rate and reservoir volume can be set independently of one another in both circuits 19, 20. The transport of the phases through the circuits 19, 20 is effected in each case by a pump 25, 26.

The tests were carried out using a commercially available membrane contactor with polypropylene hollow fibre membranes (from the company Liqui-Cel®, with Celgard® X40 hollow fibre membranes; according to the manufacturer with (wetting) properties comparable to the Celgard® 2400 flat membranes). It has an exchange surface area of 1.4 m². FIG. 5 shows a schematic and simplified representation of the mode of function of the membrane contactor 21 in a sectional view. Through the lumen-side inlet 29, the one phase is conveyed through the hollow fibre membrane 22 until it once again leaves the membrane contactor 21 at the lumen-side outlet 30. The phase fed in through the shell-side inlet 31 passes into the membrane contactor 21 and leaves the latter via the shell-side outlet 32.

In all the tests, the PEG phase was chosen as the donor phase, and the buffer phase was chosen as the acceptor phase. The equipment was operated in circulation mode in order to achieve an analytically more easily detectable change in concentration. Because of the low protein solubility in the donor phase (see above), the volume of the latter was increased to a multiple of the volume of the acceptor phase.

At the start of the test, the phases of the ATPS were prepared, as described above, and introduced into the reservoir vessels 27, 28 of the membrane equipment. The required amount of the respective protein was dissolved beforehand in the water used to dilute the PEG phase.

The circuit 20 of the non-wetting phase (acceptor or buffer phase) was first connected to the membrane contactor 21 and switched on. If required, the desired transmembrane pressure was then set up with the pressure control valve 23, a pressure of 40 mbar (measured at the contactor outlet) often being applied.

Next, the circuit 19 of the wetting phase was connected to the membrane contactor 21 and started up.

During the extraction, samples were taken from the acceptor phase and analyzed. The concentration of the proteins was determined by photospectrometry. As long as the samples for the analysis did not have to be diluted, they were returned to the acceptor phase directly after the determination. To confirm that the protein was in fact transferred, the UV/Vis spectrum of a sample was compared with that of the protein.

To minimize contamination of the equipment, the latter was cleaned after each test. The reservoir vessels 27, 28, the pumps 25, 26 and the line were first flushed with deionized water and then with acetone. The equipment was dried by blowing through compressed air. The membrane contactor 21 was flushed for at least one hour with hot water and then with acetone and 2-propanol. Thereafter, the membrane contactor 21 was dried with argon for about one hour.

The total mass transfer coefficient K was calculated from the measurement results, as described in W. Riedl, “Membrangestützte Flüssig-Flüssig-Extraktion bei der Caprolactamherstellung” [Membrane-assisted liquid-liquid extraction in caprolactam production], published by Shaker Verlag, Aachen, 2003. This parameter is a measure of the transport of substance through the membrane and thus of the extraction performance and is independent of the reservoir volume, the weighed-in quantity and the installed membrane surface.

To permit membrane-assisted extraction, Tween® 20 was added to the ATPS for wetting of the membrane by the PEG phase. Here, 27.8 g of Tween® 20 was either added before mixing in the separating funnel (tests 1 and 2) or directly into the PEG phase used for membrane extraction (1 g of Tween® 20 per 100 ml of PEG phase, tests 3-7), which makes preparation of the solutions easier.

The following table shows the results of the membrane extraction tests. In addition to general feasibility, the dependence on temperature, on fluid dynamics and on processing inside or outside of the hollow fibre membranes (lumen side/shell side) was determined.

| | | Volume PEG | Volume buffer | K |
|---|---|---|---|---|
| No. | Protein | phase [ml] | phase [ml] | [10⁻⁶ m/s] |
| 1 | Lysozyme | 1500 | 300 | 7.56 |
| 2 | Lysozyme | 1500 | 300 | 33.9 |
| 3 | Lysozyme | 1500 | 400 | 30.8 |
| 4 | Lysozyme | 1500 | 400 | 31.7 |
| 5 | Lysozyme | 1875 | 500 | 34.7 |
| 6 | Lysozyme | 1500 | 400 | 23.3 |
| 7 | BSA | 2000 | 400 | 10.8 |

The total mass transfer coefficients K achieved are between 7.56×10⁻⁶ m/s and 34.7×10⁻⁶ m/s. The measurements by Dahuron and Cussler (Protein Extraction with Hollow Fibers, AlChE Journal (34/1), 1998) are 2.0×10⁻⁶ m/s for urease and 2.8×10⁻⁷ M/s for catalase, that is to say approximately a factor of 10 lower. This is an indication that, with the process according to the invention, extraction success at least in the scope of earlier processes can be achieved, but the process is made much easier and the process stability is greatly improved.

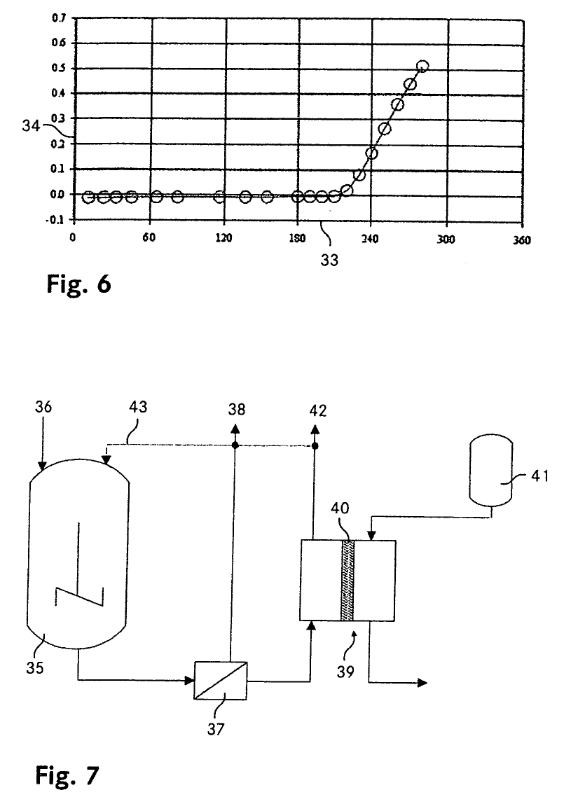

In the context of test 3 (see FIG. 6), the surfactant Tween® 20 was added to the PEG phase only after three hours of operation of the extraction equipment. During this test period, no protein could be detected in the acceptor phase. Only after the addition of Tween® 20 could the protein be detected in the acceptor phase. FIG. 6 shows the concentration profile of lysozyme in the acceptor phase before and after addition of Tween® 20. The time in minutes is indicated on the horizontal axis 33, and the vertical axis 34 shows the concentration of the component to be extracted in the acceptor phase in mg/ml. Before addition of the surfactant, the protein could not be detected in the acceptor phase, although, under the stated boundary conditions (i.e. without Tween® 20) in the agitator test, there was a detectable distribution of this useful component between the two liquid phases. It follows from this that, in membrane-assisted extraction, the two liquid phases cannot be brought into contact with one another under these conditions.

The delay in the start of extraction after addition of the surfactant (at one minute) by just under forty minutes can be explained by the fact that the added Tween® 20 first has to distribute in the test equipment in order to then completely wet the membrane pores. The successive rise in transport of substance shows that the wetting process too requires a certain time. When the wetting phase with added Tween® 20 was dropped onto a hydrophobic flat membrane, it was possible to observe that the porous membrane became clear, and was thus wetted, after just about five minutes. The extraction profile also shows that a stable transport of substance is obtained after the membrane is wetted. In this case, the total mass transfer coefficient calculated from the extraction profile is 30.8×10⁻⁶ m/s.

This shows clearly that an extraction mode is permitted only by the addition of Tween® 20. In contrast to the abovementioned earlier works (Dahuron and Cussler) in which an external pressure of the solutions was applied to both sides of the membrane in order to bring the two aqueous solutions with approximately identical wetting properties into contact via a hydrophobic membrane, in our tests only a valve was provided on the side of the buffer phase in order to set the transmembrane pressure at approximately 40 mbar, so as to be able to reliably avoid “bleeding” of an aqueous liquid phase. By contrast, this transmembrane pressure was not necessary for the process stability itself.

For toluene/water systems, it has been described earlier that, because of the essential wetting differences of this system, a stable process is possible within a wide pressure range of between almost 0 mbar and 3000 mbar (W. Riedl, “Membrangestützte Flüssig-Flüssig-Extraktion bei der Caprolactamherstellung” [Membrane-assisted liquid-liquid extraction in caprolactam production], published by Shaker Verlag, Aachen, 2003). This is because only a very low pressure is necessary to prevent escape of the wetting phase from the membrane pore, but a very high pressure has to be present in order to bring the non-wetting liquid into the membrane pore at all. Because of the wetting difference of the two aqueous phases, these results can also be transferred to the aqueous two-phase system according to the invention. Phase breakthroughs are thus to be expected only at a very high transmembrane pressure, with the result that time-consuming determination of the transmembrane pressure ought to be unnecessary in general. Compared to previously conducted works in which the transmembrane pressure had to be controlled with utmost precision in order to permit extraction, in the present case a low transmembrane pressure simply has to be applied to the buffer phase to effectively avoid bleeding of the PEG phase into the buffer phase.

In test 1, the circuit outside the hollow fibre membrane (shell side) was chosen for the PEG phase, and in test 2, for comparison, the circuit inside the hollow fibre membrane (lumen side) was chosen. In the context of test 5, the throughflow rate on the part of the PEG phase was first increased from 3.2×10⁻³ m/s to 5.0×10⁻³ m/s. Unlike the other tests, test 6 was not carried out at room temperature, but at a temperature of 36° C.

Further series of tests were carried out to examine the suitability of other aqueous two-phase systems and other surfactants.

The protein lysozyme was extracted with membrane assistance using another aqueous two-phase system, namely the PEG/dextran/water system. The test was conducted in the same way as was described above for the PEG/phosphate buffer system for tests 2 to 4. Here once again, extraction was able to be successfully performed with process stability only upon addition of the surfactant Tween® 20, as is shown by the following profile of the concentration of lysozyme in the buffer phase (acceptor phase):

| Time | c |
|---|---|
| (min) | (mg/ml) |
| 0 | 0.0000 |
| 10 | 0.0020 |
| 30 | 0.0416 |
| 60 | 0.2594 |
| 75 | 0.3890 |
| 93 | 0.5046 |
| 105 | 0.5568 |
| 125 | 0.6562 |

The total mass transfer coefficient K derived from these values is, at 19.9×10⁻⁶ m/s, of the same order of magnitude as the coefficient determined in the first series of tests using the PEG/phosphate buffer system. The application of the process according to the invention is therefore not bound to one defined aqueous two-phase system.

Next, the influence of different surfactants on the wetting properties of the phases of different aqueous two-phase systems was investigated. In addition to the already described Tween® 20, the following surfactants were now also examined: Triton® X-100 (CAS No. 9002-93-1) and Brij® 35 P (CAS No. 9002-92-0). In addition to the already mentioned ATPS, the PEG/sodium sulphate system was now also examined. The aqueous two-phase systems were prepared in a separating funnel.

From the systems thus prepared (with and without addition of the abovementioned surfactants), drop tests were then carried out on a microporous, hydrophobic polymer membrane (Celgard ® 2400, cf. the preceding wetting tests). The aim here was to establish whether the solutions to which the abovementioned surfactants were added run on the membrane and penetrate into the membrane matrix (membrane pores) and thereby are able to wet the membrane, or whether they form a droplet on the membrane and thus, like the PEG/phosphate buffer ATPS system without added surfactant, do not wet the membrane.

Compared to the abovementioned tests with exact measurement of the contact angles, the behaviour of the liquids when dropped onto the membrane was in this case evaluated purely visually. This by itself permitted a clear distinction between those cases in which a solution wetted the surface of the membrane and those cases in which the membrane was not wetted.

| | | | Liquid phase wets |
|---|---|---|---|
| | | C_{T} | hydrophobic |
| ATPS | Surfactant | (wt. %) | membrane |
| PEG/phosphate buffer | — | — | no; drop formation |
| PEG/phosphate buffer | Tween ® 20 | 1.3 | yes |
| PEG/phosphate buffer | Triton ® X-100 | 1.3 | yes |
| PEG/phosphate buffer | Brij ® 35 P | 0.1 | yes |
| PEG/Na₂SO₄ | — | — | no; drop formation |
| PEG/Na₂SO₄ | Tween ® 20 | 1.3 | yes |
| PEG/Na₂SO₄ | Triton ® X-100 | 1.3 | yes |
| PEG/Na₂SO₄ | Brij ® 35 P | 0.1 | yes |
| PEG/dextran | — | — | no; drop formation |
| PEG/dextran | Tween ® 20 | 0.9 | yes |
| PEG/dextran | Triton ® X-100 | 0.9 | yes |
| PEG/dextran | Brij ® 35 P | 0.06 | yes |

As the table shows, addition of even low concentrations of surfactants in all the ATPS means that the liquid phase to which the surfactant was added wets the test membrane. Without addition of a surfactant, by contrast, the ATPS do not wet the membrane.

Accordingly, the important difference for application of the process according to the invention, i.e. the important difference in the wettability of the two liquid phases, is also provided with the surfactants Triton® X-100 and Brij® 35 P and is thus not confined to a specific surfactant.

Industrial Implementation of the Process

An example of the industrial implementation of the process according to the invention is outlined below. This is shown schematically in FIG. 7. Here, the membrane-assisted extraction is employed to extract (“harvest”) the biological useful products produced in a fermentation process.

In a fermenter 35 in which a reaction based on aqueous solutions has been completed, the required amounts of concentrated buffer solution, concentrated PEG solution, Tween® 20 and water are added (arrow 36) in order to obtain a PEG phase of the corresponding aqueous two-phase system. By means of a preceding process step of cleaning, e.g. ultrafiltration or microfiltration, the cell residues in a corresponding device 37 are now removed from the resulting donor phase and transported away (arrow 38). The clarified donor phase is pumped in countercurrent through a suitable membrane contactor 39 which, on the other side of the membrane 40, is already flushed with the prepared buffer acceptor phase. With an optimal configuration of the membrane contactor 39, a high degree of accumulation of the biological material to be extracted takes place in the buffer phase, which is pumped from a reservoir vessel 41 through the membrane contactor 39. The depleted PEG phase is transported away after passage through the membrane contactor (arrow 42).

If the cells do not have to be destroyed in the process, circulation techniques are used in separating the cells off. Also, if the biological reaction is not stopped by the ATPS components, a continuous circulation process can be effected in which the concentrated cell broth together with the depleted PEG phase from the membrane contactor 39 and with fresh educts is returned to the fermenter 35 (arrow 43).

The invention is not limited to the illustrative embodiments described. The results of the extensive series of tests and of the comparison tests show that the invention can be carried out on a large number of ATPS and that a great many surface-active agents can be used to modify at least one of the phases. The process can be used for extraction of a wide variety of biological materials.

Many variations are also possible in the conduct of the process and its industrial implementation, especially as regards the choice of membrane contactor used and the test set-up. For example, additional process steps for treatment of the two phases can easily be carried out before or after the actual extraction. The process can also be carried out in co-current mode, or strengthening processes can be used.

It may be stated in conclusion that the invention makes available a process belonging to the technical field mentioned in the introduction and permitting extraction of biological material, said process being easy to carry out and being able to be used on an industrial scale.

1. Process for extraction of biological material, in particular of proteins or peptides, from a first aqueous solution to a second aqueous solution, a porous membrane being arranged between the aqueous solutions, characterized in that at least one of the aqueous solutions is modified by addition of a biocompatible and surface-active agent in such a way that, in relation to the membrane used, it has a different wettability than the other aqueous solution.

2. Process according to claim 1, characterized in that the at least one of the aqueous solutions is modified in such a way that it wets the membrane used and can penetrate into the pores of the latter, whereas the other aqueous solution does not wet the membrane and thus does not penetrate into the pores of the latter.

3. Process according to claim 1 or claim 2, characterized in that the one of the aqueous solutions is modified in such a way that a difference between a first contact angle between the first aqueous solution and the surface of the membrane and a second contact angle between the second aqueous solution and the surface of the membrane is at least 5°, preferably at least 10°.

4. Process according to claim 1, characterized in that the porous membrane is hydrophobic.

5. Process according to claim 4, characterized in that the one of the aqueous solutions is modified in such a way that a contact angle between the modified aqueous solution and a surface of the membrane is less than 90°, preferably less than 50°.

6. Process according to claim 1, characterized in that the agent is a surfactant.

7. Process according to claim 6, characterized in that the agent is chosen from one of the following agents: Tween-20, Triton X-100, Brij 35 P.

8. Process according to claim 6 or claim 7, characterized in that the one of the aqueous solutions is modified in such a way that a concentration of the surfactant in the modified aqueous solution is at most 2.5% by weight, preferably less than 1.0% by weight.

9. Process according to claim 1, characterized in that the porous membrane is hydrophilic, and in that the one of the aqueous solutions is modified in such a way that a contact angle between the modified aqueous solution and a surface of the membrane is at least 90°.

10. Process according to claim 1, characterized in that the agent chosen for modifying at least the one aqueous solution is an agent which, in comparison with the other aqueous solution, accumulates very much preferentially in the aqueous solution that is to be modified.

11. Process according to claim 1, characterized in that the membrane is a porous flat membrane, capillary membrane or hollow fibre membrane.

12. Process according to claim 1, characterized in that a maximum pore diameter of the membrane is 2 μm.

13. Process according to claim 1, characterized in that one of the aqueous solutions contains a polymer, in particular PEG (polyethylene glycol) or dextran, and in that a salt or a phosphate buffer is preferably dissolved in the other of the solutions.

14. Process according to claim 1, characterized in that the first aqueous solution contains a first polymer, and in that the second aqueous solution contains a second polymer, the polymers being water-soluble and being immiscible or only slightly miscible with one another, and the two polymers preferably being polyethylene glycol (PEG) and dextran.

15. Process according to claim 1, characterized in that, before or after the extraction, at least one of the aqueous solutions has its physical, chemical and/or thermodynamic properties altered by means of distillation, by means of membrane processes, flocculation and/or by other suitable separation processes.

16. Use of a biocompatible agent in a process, in particular according to claim 1, for extraction of biological material from a first aqueous solution to a second aqueous solution, a porous membrane being arranged between the aqueous solutions, characterized in that at least one of the aqueous solutions is modified by addition of the agent in such a way that, in relation to the membrane used, it has a different wettability than the other aqueous solution.
